(19) United States
(12) Patent Application Publication
(10) Pub. No.: US 2008/0019927 A1
(43) Pub. Date: Jan. 24, 2008

(54) Compositions and methods for dermally treating neuropathy with minoxidil

(76) Inventors: Jie Zhang, Salt Lake City, UT (US); Kevin S. Warner, West Jordan, UT (US); Sanjay Sharma, Sandy, UT (US)

Correspondence Address: THORPE NORTH & WESTERN, LLP., 8180 SOUTH 700 EAST, SUITE 350, SANDY, UT, 84070, US

(21) Appl. No.: 11/888,905

(22) Filed: Aug. 1, 2007

Related U.S. Application Data
(63) Continuation-in-part of application No. 11/640,139, filed on Dec. 14, 2006.
(63) Continuation-in-part of application No. 11/146,917, filed on Jun. 6, 2005.
(63) Continuation-in-part of application No. 11/640,135, filed on Dec. 14, 2006.
(63) Continuation-in-part of application No. 11/146,917, filed on Jun. 6, 2005.
(60) Provisional application No. 60/750,637, filed on Dec. 14, 2005.
(60) Provisional application No. 60/750,519, filed on Dec. 14, 2005.
(60) Provisional application No. 60/577,536, filed on Jun. 7, 2004.
(60) Provisional application No. 60/750,637, filed on Dec. 14, 2005.
(60) Provisional application No. 60/577,536, filed on Jun. 7, 2004.

(51) Int. Cl. A61K 9/12; A61K 31/165; A61K 31/415; A61K 31/445; A61P 17/00; A61K 31/505; A61K 31/56

(52) U.S. Cl. 424/45; 514/182; 514/275; 514/330; 514/401; 514/617

(57) ABSTRACT

The present invention is drawn to adhesive solidifying formulations containing minoxidil that can be used for treating neuropathies including diabetic neuropathy. The formulation can include an amount of minoxidil, a solvent vehicle, and a solidifying agent. The solvent vehicle can include a volatile solvent system including at least one volatile solvent, and a non-volatile solvent system including at least one non-volatile solvent capable of facilitating the delivery of the minoxidil at therapeutically effective rates over a sustained period of time. The formulation can have a viscosity suitable for application to a skin surface prior to evaporation of the volatile solvents system. When applied to the skin, the formulation can form a solidified layer after at least a portion of the volatile solvent system is evaporated.

This application is a continuation-in-part of U.S. patent application Ser. No. 11/640,139 filed on Dec. 14, 2006, which claims the benefit of U.S. Provisional Application Nos. 60/750,637 and 60/750,519 filed on Dec. 14, 2005 and is also a continuation-in-part of U.S. application Ser. No. 11/146,917 filed on Jun. 6, 2005, which claims the benefit of U.S. Provisional Application No. 60/577,536 filed on Jun. 7, 2004, each of which is incorporated herein by reference; and additionally, this application is a continuation-in-part of U.S. patent application Ser. No. 11/640,135 filed on Dec. 14, 2006, which claims the benefit of U.S. Provisional Application No. 60/750,637 filed on Dec. 14, 2005 and is also a continuation-in-part of U.S. application Ser. No. 11/146,917 filed on Jun. 6, 2005, which claims the benefit of U.S. Provisional Application No. 60/577,536 filed on Jun. 7, 2004, each of which is incorporated herein by reference.

### FIELD OF THE INVENTION

The present invention relates generally to formulations and methods developed for the use of minoxidil, such as to treat neuropathies and the pain associated therewith. More particularly, the present invention relates to adhesive solidifying formulations having a viscosity suitable for application to a skin surface, and which form a sustained drug-delivering adhesive solidified layer on the skin.

### BACKGROUND OF THE INVENTION

Neuropathies can be caused by various underlying diseases, such as viral infections and diabetes. Diabetic neuropathy is often suffered by diabetic patients and can be accompanied by a variety of symptoms including pain. Topical products, such as creams or patches containing appropriate drugs, may be used to control neuropathy; however, patches and traditional semisolid formulations such as creams and ointments both have significant shortcomings. Semisolid formulations usually contain solvent(s), such as water and ethanol, which are volatile and thus evaporate shortly after application. The evaporation of such solvents can cause significant decrease or even termination of dermal drug delivery, which can be undesirable in many cases. Additionally, semisolid formulations are often “rubbed into” the skin, which does not necessarily mean the drug formulation is actually delivered into the skin. Instead, this phrase often means that a very thin layer of the drug formulation is applied onto the surface of the skin. Such thin layers of traditional semisolid formulations applied to the skin may not contain sufficient quantity of the active drug to achieve sustained delivery over long periods of time, which can be desirable in treating neuropathy. Additionally, traditional semisolid formulations are often subject to unintentional removal due to contact with objects such as clothing, which may compromise the sustained delivery and/or undesirably soil clothing.

A patch containing an appropriate drug can be used to treat neuropathy. However, subjects often have to cut the patch to fit the shape and size of the skin area to be treated, which is inconvenient. Another shortcoming of patches is that they are usually neither sufficiently stretchable nor flexible for every application location. If the patch is applied on a skin area that is significantly stretched during body movements, such as joints and muscles, separation between the patch and skin may occur, thereby compromising the delivery of the drug. In addition, a patch on a skin surface may hinder the expansion of the skin during body movements and cause discomfort and/or aggravate pain. For these additional reasons, patches are not ideal dosage forms for skin areas subject to expansion and stretching during body movements.

In view of these and other shortcomings, it would be desirable to provide systems, formulations, and/or methods for treating neuropathy that i) can provide sustained drug delivery over long periods of time; ii) are not vulnerable to unintentional removal by contact with either clothing, other objects, or with other people for the duration of the application time; iii) can be applied to a skin area subject to stretching and expansion without causing discomfort or poor contact to skin; and/or iv) can be easily removed after application and use.

### SUMMARY OF THE INVENTION

It has been recognized that it would be advantageous to provide topical neuropathy treatment formulations, systems, and/or methods in the form of adhesive solidifying compositions or formulations having a viscosity suitable for application to a skin surface as a layer, and which form aminoxidil-delivering solidified layer on the skin that can be easily peelable or removable after use.

In accordance with this, a formulation for treating neuropathy can comprise an amount of minoxidil suitable for treating neuropathy and pain associated therewith, a solvent vehicle, and a solidifying agent. The solvent vehicle can include a volatile solvent system comprising at least one volatile solvent, and a non-volatile solvent system comprising at least one non-volatile solvent. The non-volatile solvent system facilitates dermal delivery of the minoxidil at a therapeutically effective rate over a sustained period of time. The formulation can have a viscosity suitable for application and adhesion to a skin surface as a layer prior to evaporation of the volatile solvent system. The formulation applied to the skin surface can form a solidified layer after at least partial evaporation of the volatile solvent system. Further, the minoxidil can continue to be delivered at the therapeutically effective rate after the volatile solvent system is at least substantially evaporated.

In another embodiment, a method for treating neuropathy can comprise the step of applying a layer of an adhesive formulation to a skin surface of a subject. The formulation can comprise an amount of minoxidil suitable for treating neuropathy and pain associated therewith, a solvent vehicle, and a solidifying agent. The solvent vehicle can comprise a volatile solvent system including at least one volatile solvent, and a non-volatile solvent system including at least one non-volatile solvent. The non-volatile solvent system facilitates dermal delivery of the minoxidil at a therapeutically effective rate over a sustained period of time. The formulation can have a viscosity suitable for application and adhesion to a skin surface prior to evaporation of the volatile solvent system. Additional steps include solidifying the formulation to form a solidified layer on the skin surface by at least partial evaporation of the volatile solvent system; and dermally delivering the minoxidil from the solidified layer to the subject at therapeutically effective rates over a sustained period of time to treat neuropathy.

In another embodiment, a solidified layer for treating neuropathy can comprise an amount of minoxidil suitable for treating neuropathy, a non-volatile solvent system suitable for the minoxidil, and a solidifying agent. The solidified layer can have sufficient elasticity, flexibility, and adhesion to the skin so that it is not separated from the skin even if the skin surface is stretched or bent during a subject's normal daily activities. For example, the solidified layer can be stretchable by 5%, in one direction without cracking, breaking, and/or separating from a skin surface to which the layer is applied.

Additional features and advantages of the invention will be apparent from the following detailed description which illustrates, by way of example, features of the invention.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a drug” includes reference to one or more of such compositions.

“Skin” is defined to include human skin (intact, diseased, ulcerous, or broken), finger and toe nail surfaces, and mucosal surfaces that are usually at least partially exposed to air such as lips, genital and anal mucosa, and nasal and oral mucosa.

The term “neuropathy” refers to any and all types of neuropathy regardless of the cause. Examples of specific neuropathies for which the formulations and methods of the present invention can be used include diabetic neuropathies and viral caused neuropathies. The treatment of neuropathy as described herein refers to the alleviation or elimination of at least one of the symptoms associated with neuropathy, including but not limited neuropathic pain.

The term “drug(s)” or “drug(s) suitable for treating neuropathy” refers to any bioactive agent that is applied to, into, or through the skin for preventing and/or treating neuropathy and pain associated therewith. The present application focuses primarily on the use of certain minoxidil formulations to treat neuropathy, especially neuropathy caused by diabetes. Minoxidil can comprise about 0.1 wt % to about 30 wt % of such formulations. In one embodiment, minoxidil can comprise about 0.5 wt % to about 10 wt % of the formulation. In another embodiment, the minoxidil can comprise about 2 wt % to about 6 wt % of the formulation. Optionally, other drugs can be co-administered with the minoxidil. As described herein, the solidified layers of the present invention form when at least a portion of the volatile solvents in the applied formulation has evaporated. As such, it is understood that the wt % of minoxidil in a solidified layer will increase as a solidified layer is formed and more volatile solvent evaporates.

When the formulations of the present invention form solidified layers, such layers can be capbable of delivering minoxidil at a transdermal flux rate of at least 0.02 μg/cm²/hour across human skin. In one embodiment, the solidified layer transdermally delivers the minoxidil at a flux rate of about 0.1 μg/cm²/hour to about 5.0 μg/cm²/hour across human skin. In another embodiment, the solidified layer transdermally delivers the minoxidil at a flux rate of about 0.4 μg/cm²/hour to about 4.0 μg/cm²/hour across human skin.

The formulations of the present invention can include minoxidil alone or in combination with other drugs suitable for treating neuropathy and pain associated therewith. Non-limiting examples of such drugs include local anesthetics including lidocaine, bupivacaine, ropivacaine, and tetracaine; steroids including dexamethasone; alpha-2 agonists including clonidine; tricyclic anti-depressants including amitriptyline, anticonvulsants, N-methyl-D-aspartate (NMDA) antagonists including dextromethorphan, memantine, amantadine, ketamine, methadone, dextropropoxyphene, and ketobemidone; antiviral drugs including acyclovir, penciclovir, famciclovir, valacyclovir steroids; 5-HT2A receptor antagonist including ketanserin; or combinations thereof.

The phrases “dermal drug delivery” or “dermal delivery of drug(s)” shall include both transdermal and topical drug delivery, and includes the delivery of drug(s) to, through, or into the skin. “Transdermal delivery” of drug can be targeted to skin tissues just under the skin, regional tissues or organs under the skin, systemic circulation, and/or the central nervous system.

The term “flux” such as in the context of “dermal flux” or “transdermal flux,” respectively, refers to the quantity of the drug permeated into or across skin per unit area per unit time, measured in either an in vitro system commonly used in the art (such as that described in Example 1), or measured in live human subjects. A typical unit of flux is microgram per square centimeter per hour. One way to measure flux is to place the formulation on a known skin area of a human volunteer and measure how much drug can permeate into or across skin within certain time constraints. Various methods (in vivo methods) might be used for the measurements as well. The method described in Example 1 or other similar method (in vitro methods) can also be used to measure flux. Although an in vitro method uses human epidermal membrane obtained from a cadaver, or freshly separated skin tissue from hairless mice rather than measure drug flux across the skin using human volunteers, it is generally accepted by those skilled in the art that results from a properly designed and executed in vitro test can be used to estimate or predict the results of an in vivo test with reasonable reliability. Therefore, “flux” values referenced in this patent application can mean that measured by either in vivo or in vitro methods.

The term “flux-enabling” with respect to the non-volatile solvent system (or solidified layer including the same) refers to a non-volatile solvent system (including one or more non-volatile solvents) selected or formulated specifically to be able to provide therapeutically effective flux for a particular drug(s). For topically or regionally delivered drugs, a flux enabling non-volatile solvent system is defined as a non-volatile solvent system which, alone without the help of any other ingredients, is capable of delivering therapeutic effective levels of the drug across, onto or into the subject's skin when the non-volatile solvent system is saturated with the drug. For systemically targeted drugs, a flux enabling non-volatile solvent system is a non-volatile solvent system that can provide therapeutically sufficient daily doses over 24 hours when the non-volatile solvent system is saturated with the drug and is in full contact with the subject's skin with no more than 500 cm² contact area. Preferably, the contact area for the non-volatile solvent system is no more than 100 cm². Testing using this saturated drug-in-solvent state can be used to measure the maximum flux-generating ability of a non-volatile solvent system. To determine flux, the drug solvent mixture needs to be kept on the skin for a clinically sufficient amount of time. In reality, it may be difficult to keep a liquid solvent on the skin of a human volunteer for an extended period of time. Therefore, an alternative method to determine whether a solvent system is “flux-enabling” is to measure the in vitro drug permeation across the hairless mouse skin or human cadaver skin using the apparatus and method described in Example 1. This and similar methods are commonly used by those skilled in the art to evaluate permeability and feasibility of formulations. Alternatively, whether a non-volatile solvent system is flux-enabling can be tested on the skin of a live human subject with means to maintain the non-volatile solvent system with saturated drug on the skin, and such means may not be practical for a product. For example, the non-volatile solvent system with saturated drug can be soaked into an absorbent fabric material which is then applied on the skin and covered with a protective membrane. Such a system is not practical as a pharmaceutical product, but is appropriate for testing whether a non-volatile solvent system has the intrinsic ability to provide sufficient drug flux, or whether it is flux-enabling.

It is also noted that once the formulation forms a solidified layer, the solidified layer can also be “flux enabling” for the drug while some of the non-volatile solvents remain in the solidified layer, even after the volatile solvents (including water) have been substantially evaporated.

For minoxidil, a non-volatile solvent system would be “flux enabling” if it is capable of generating a flux of at least about 0.02 mcg/cm²/hour in a setup same or similar to that described in Example 1, or in live human subjects. For lidocaine base, a non-volatile solvent system would be “flux enabling” if it is capable of generating a flux of at least about 5 mcg/cm²/hour, and more typically 20 mcg/cm²/hour, in a setup same or similar to that described in Example 1, or in live human subjects. For tetracaine and ropivacaine bases, a non-volatile solvent system would be “flux enabling” if it is capable of generating a flux of at least about 5 mcg/cm²/hour in a setup same or similar to that described in Example 1, or in live human subjects. For amitriptyline, a non-volatile solvent system would be “flux enabling” if it is capable of generating a flux of about at least 5 mcg/cm²/hour in a setup same or similar to that described in Example 1, or in live human subjects.

The phrase “effective amount,” “therapeutically effective amount,” “therapeutically effective rate(s),” or the like, as it relates to a drug, refers to sufficient amounts or delivery rates of minoxidil which achieves any appreciable level of therapeutic results in treating neuropathy. It is understood that “appreciable level of therapeutic results” may or may not meet any government agencies' efficacy standards for approving the commercialization of a product. It is understood that various biological factors may affect the ability of a substance to perform its intended task. Therefore, an “effective amount,” “therapeutically effective amount,” or “therapeutically effective rate(s)” may be dependent in some instances on such biological factors to some degree. However, for each drug, there is usually a consensus among those skilled in the art on the range of doses or fluxes that are sufficient in most subjects. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

“Therapeutically effective flux” refers to the permeation flux of minoxidil that delivers sufficient amount of minoxidil into or across the skin to be clinically beneficial in that some of the patient population can obtain some degree of benefit from the drug flux. It does not necessarily mean that most of the patient population can obtain some degree of benefit or the benefit is high enough to be deemed “effective” by relevant government agencies or the medical profession. More specifically, for drugs that target skin or regional tissues or organs close to the skin surface (such as joints, certain muscles, or tissues/organs that are at least partially within 5 cm of the skin surface), “therapeutically effective flux” refers to the drug flux that can deliver a sufficient amount of the drug into the target tissues within a clinically reasonable amount of time.

It should be noted that “flux-enabling non-volatile solvent,” “flux-enabling, plasticizing non-volatile solvent,” or “high flux-enabling non-volatile solvent” can be a single chemical substance or a mixture of two or more chemical substances, as can be seen in the Examples.

The term “adhesion” or “adhesive” when referring to a solidified layer herein refers to sufficient adhesion between the solidified layer and the skin so that the layer does not fall off the skin during intended use on most subjects. Thus, “adhesive” or the like when used to describe the solidified layer means the solidified layer is adhesive to the body surface to which the initial formulation layer was originally applied (before the evaporation of the volatile solvent(s)). In one embodiment, it does not mean the solidified layer is adhesive on the opposing side. In addition, it should be noted that whether a solidified layer can adhere to a skin surface for the desired extended period of time partially depends on the condition of the body surface. For example, excessively sweating or oily skin, or oily substances on the skin surface may make the solidified layer less adhesive to the skin. Therefore, the adhesive solidified layer of the present invention may not be able to maintain perfect contact with the body surface and deliver the drug over a sustained period of time for every subject under any conditions on the body surface. A standard is that it maintains good contact with most of the body surface, e.g. 70% of the total area, over the specified period of time for most subjects under normal conditions of the body surface and external environment.

The terms “flexible,” “elastic,” “elasticity,” or the like, as used herein refer to sufficient elasticity of the solidified layer so that it is not broken if it is stretched in at least one direction by up to about 5%, and often to about 10% or even greater. For example, a solidified layer that exhibits acceptably elasticity and adhesion to skin can be attached to human skin over a flexible skin location, e.g., elbow, finger, wrist, neck, lower back, lips, knee, etc., and will remain substantially intact on the skin upon stretching of the skin. It should be noted that the solidified layers of the present invention do not necessarily have to have any elasticity in some embodiments.

The term “peelable,” when used to describe the solidified layer, means the solidified layer can be lifted from the skin surface in one large piece or several large pieces, as opposed to many small pieces or crumbs.

The term “sustained” relates to therapeutically effective rates of dermal minoxidil delivery for a continuous period of time of at least 30 minutes, and in some embodiments, periods of time of at least about 2 hours, 4 hours, 8 hours, 12 hours, 24 hours, or longer.

The use of the term “substantially” when referring to the evaporation of the volatile solvents means that a majority of the volatile solvents which were included in the initial formulation have evaporated. Similarly, when a solidified layer is said to be “substantially devoid” of volatile solvents, including water, the solidified layer has less than 10 wt %, and preferably less than 5 wt %, of the volatile solvents in the solidified layer as a whole.

“Volatile solvent system” can be a single solvent or a mixture of solvents that are volatile, including water and solvents that are more volatile than water. Non-limiting examples of volatile solvents that can be used in the present invention include iso-amyl acetate, denatured alcohol, methanol, ethanol, isopropyl alcohol, water, propanol, C4-C6 hydrocarbons, butane, isobutene, pentane, hexane, acetone, chlorobutanol, ethyl acetate, fluoro-chloro-hydrocarbons, turpentine, methyl ethyl ketone, methyl ether, hydrofluorocarbons, ethyl ether, 1,1,1,2 tetrafluorethane 1,1,1,2,3,3,3-heptafluoropropane, 1,1,1,3,3,3 hexafluoropropane, or combinations thereof.

“Non-volatile solvent system” can be a single solvent or mixture of solvents that are less volatile than water. It can also contain substances that are solid or liquid at room temperatures, such as pH or ion-pairing agents. After evaporation of the volatile solvent system, most of the non-volatile solvent system should remain in the solidified layer for an amount of time sufficient to dermally delivery a given drug to, into, or through the skin of a subject at a sufficient flux for a period of time to provide a therapeutic effect. In some embodiments, in order to obtain desired permeability for an active drug and/or compatibility with solidifying agents or other ingredients of the formulation, a mixture of two or more non-volatile solvents can be used to form the non-volatile solvent system. In one embodiment, the combination of two or more non-volatile solvents to form a solvent system provides a higher transdermal flux for a drug than the flux provided for the drug by each of the non-volatile solvents individually. The non-volatile solvent system may also serve as a plasticizer of the solidified layer, so that the solidified layer is elastic and flexible.

The term “solvent vehicle” describes compositions that include both a volatile solvent system and non-volatile solvent system. The volatile solvent system is chosen so as to evaporate from the adhesive peelable formulation quickly to form a solidified layer, and the non-volatile solvent system is formulated or chosen to substantially remain as part of the solidified layer after volatile solvent system evaporation so as to provide continued delivery of the drug. Typically, the drug can be partially or completely dissolved in the solvent vehicle or formulation as a whole. Likewise, the drug can also be partially or completely solubilizable in the non-volatile solvent system once the volatile solvent system is evaporated. Formulations in which the drug is only partially dissolved in the non-volatile solvent system after the evaporation of the volatile solvent system have the potential to maintain longer duration of sustained delivery, as the undissolved drug can dissolve into the non-volatile solvent system as the dissolved drug is being depleted from the solidified layer during drug delivery.

“Adhesive solidifying formulation” or “solidifying formulation” refers to a composition that has a viscosity suitable for application to a skin surface prior to evaporation of its volatile solvent(s), and which can become a solidified layer after evaporation of at least a portion of the volatile solvent(s). The solidified layer, once formed, can be very durable. In one embodiment, once solidified on a skin surface, the formulation can form a peel. The peel can be a soft, coherent solid that can be removed by peeling large pieces from the skin relative to the size of the applied formulation, and often, can be peeled from the skin as a single piece. The application viscosity is typically more viscous than a water-like liquid, but less viscous than a soft solid. Examples of preferred viscosities include materials that have consistencies similar to pastes, gels, ointments, and the like, e.g., viscous liquids that flow but are not subject to spilling. Thus, when a composition is said to have a viscosity “suitable for application” to a skin surface, this means the composition has a viscosity that is high enough so that the composition does not substantially run off the skin after being applied to skin, but also has a low enough viscosity so that it can be easily spread onto the skin. A viscosity range that meets this definition can be from about 100 cP to about 3,000,000 cP (centipoises), and more preferably from about 1,000 cP to about 1,000,000 cP.

In some embodiments of the present invention, it may be desirable to add an additional agent or substance to the formulation so as to provide enhanced or increased adhesive characteristics. The additional adhesive agent or substance can be an additional non-volatile solvent or an additional solidifying agent. Non-limiting examples of substances which might be used as additional adhesion enhancing agents include copolymers of methylvinyl ether and maleic anhydride (Gantrez polymers), polyethylene glycol and polyvinyl pyrrolidone, gelatin, low molecular weight polyisobutylene rubber, copolymer of acrylsan alkyl/octylacrylamido (Dermacryl 79), and various aliphatic resins and aromatic resins.

The terms “washable,” “washing,” or “removed by washing” when used with respect to the adhesive formulations of the present invention refers to the ability of the adhesive formulation to be removed by the application of a washing solvent using a normal or medium amount of washing force. The required force to remove the formulations by washing should not cause significant skin irritation or abrasion. Generally, gentle washing force accompanied by the application of an appropriate washing solvent is sufficient to remove the adhesive formulations disclosed herein. The solvents which can be used for removing by washing the formulations of the present invention are numerous, but preferably are chosen from commonly acceptable solvents including the volatile solvents listed herein. Preferred washing solvents do not significantly irritate human skin and are generally available to the average subject. Examples of washing solvents include but are not limited to water, ethanol, methanol, isopropyl alcohol, acetone, ethyl acetate, propanol, or combinations thereof. In aspect of the invention the washing solvents can be selected from the group consisting of water, ethanol, isopropyl alcohol or combinations thereof. Surfactants can also be used in some embodiments.

Acceptable lengths of time when referring to “drying time” refers to the time it takes for the formulation to form a non-messy solidified surface after application on skin under standard skin and ambient conditions, and with standard testing procedure. It is noted that the word “drying time” in this application does not mean the time it takes to completely evaporate off the volatile solvent(s). Instead, it means the time it takes to form the non-messy solidified surface as described above.

“Standard skin” is defined as dry, healthy human skin with a surface temperature of between about 30° C. to about 36° C. Standard ambient conditions are defined by the temperature range of from 20° C. to 25° C. and a relative humidity range of from 20% to 80%. The term “standard skin” in no way limits the types of skin or skin conditions on which the formulations of the present invention can be used. The formulations of the present invention can be used to treat all types of “skin,” including undamaged (standard skin), diseased skin, or damaged skin. Although skin conditions having different characteristics can be treated using the formulations of the present invention, the use of the term “standard skin” is used merely as a standard to test the compositions of the varying embodiments of the present invention. As a practical matter, formulations that perform well (e.g., solidify, provide therapeutically effective flux, etc.) on standard skin can also perform well diseased or damaged skin.

The “standard testing procedure” or “standard testing condition” is as follows: To standard skin at standard ambient conditions is applied an approximately 0.1 mm layer of the adhesive solidifying formulation and the drying time is measured. The drying time is defined as the time it takes for the formulation to form a non-messy surface such that the formulation does not lose mass by adhesion to a piece of 100% cotton cloth pressed onto the formulation surface with a pressure of between about 5 and about 10 g/cm² for 5 seconds.

“Solidified layer” describes the solidified or dried layer of an adhesive solidifying formulation after at least a portion of the volatile solvent system has evaporated. The solidified layer remains adhered to the skin, and is preferably capable of maintaining good contact with the subject's skin for substantially the entire duration of application under standard skin and ambient conditions. The solidified layer also preferably exhibits sufficient tensile strength so that it can be peeled off the skin at the end of the application in one piece or several large pieces (as opposed to a layer with weak tensile strength that breaks into many small pieces or crumbles when removed from the skin).

As used herein, a plurality of drugs, compounds, and/or solvents may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With these definitions in mind, a formulation for treating neuropathy can comprise an amount of minoxidil suitable for treating neuropathy or pain associated therewith, a solvent vehicle, and a solidifying agent. The solvent vehicle can include a volatile solvent system comprising at least one volatile solvent, and a non-volatile solvent system comprising at least one non-volatile solvent. The non-volatile solvent system facilitates dermal delivery of the minoxidil at a therapeutically effective rate over a sustained period of time. The formulation can have a viscosity suitable for application and adhesion to a skin surface as a layer prior to evaporation of the volatile solvent system. The formulation applied to the skin surface can form a solidified layer after at least partial evaporation of the volatile solvent system. Further, the minoxidil can continue to be delivered at the therapeutically effective rate after the volatile solvent system is at least substantially evaporated.

In another embodiment, a method for treating neuropathy can comprise the step of applying a layer of an adhesive formulation to a skin surface of a subject suffering from neuropathy. The solidifying formulation can comprise an amount of minoxidil suitable for treating neuropathy, a solvent vehicle, and a solidifying agent. The solvent vehicle can comprise a volatile solvent system including at least one volatile solvent, and a non-volatile solvent system including at least one non-volatile solvent. The non-volatile solvent system facilitates dermal delivery of the minoxidil at a therapeutically effective rate over a sustained period of time. The formulation can have a viscosity suitable for application and adhesion to a skin surface prior to evaporation of the volatile solvent system. Additional steps include solidifying the formulation to form a solidified layer on the skin surface by at least partial evaporation of the volatile solvent system; and dermally delivering the minoxidil from the solidified layer to the subject at therapeutically effective rates over a sustained period of time to treat the neuropathy.

In another embodiment, a solidified layer for treating neuropathy can comprise an amount of minoxidil suitable for treating neuropathy, a non-volatile solvent system suitable for the minoxidil, and a solidifying agent. The solidified layer can have sufficient elasticity, flexibility, and adhesion to the skin so that it is not separated from the skin even if the skin surface is stretched or bent during a subject's normal daily activities. For example, the solidified layer can be stretchable by 5% in one direction without cracking, breaking, and/or separating from a skin surface to which the layer is applied.

Thus, the present invention is related to novel formulations, methods, and solidified layers that are typically related to initial formulations of semi-solids (including creams, gels, pastes, ointments, and other viscous liquids), which can be easily applied onto the skin as a layer, and can quickly (from 15 seconds to minutes under standard skin and ambient conditions) to moderately quickly (from 4 to 15 minutes under standard skin and ambient conditions) change into a solidified layer, e.g., a coherent and soft solid layer, for drug delivery for treating neuropathy. A solidified layer thus formed is capable of delivering a therapeutically effective amount of minoxidil to the skin, into the skin, across the skin, etc., at therapeutically effective rates, over a sustained period of time, e.g., hours to tens of hours, so that most of the drug delivery occurs after the solidified layer is formed. Additionally, the solidified layer typically adheres to the skin, but has a solidified, minimally-adhering, outer surface which is formed relatively soon after application and which does not substantially transfer to or otherwise soil clothing or other objects that a subject is wearing or that the solidified layer may inadvertently contact. The solidified layer can preferably also be formulated such that it is highly flexible and stretchable, and thus capable of maintaining good contact with a skin surface, even if the skin is stretched during body movement, such as at a knee, finger, elbow, or other joints.

In selecting the various components that can be used, e.g. solvent vehicle of volatile solvent system and non-volatile solvent system, solidifying agent(s), etc., various considerations may be relevant. For example, the volatile solvent system can be selected from pharmaceutically or cosmetically acceptable solvents known in the art. In one embodiment of the present invention, the volatile solvent system can include ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone or combinations thereof. In another embodiment of the present invention, the volatile solvent system can include iso-amyl acetate, denatured alcohol, methanol, propanol, isobutene, pentane, hexane, chlorobutanol, turpentine, cytopentasiloxane, cyclomethicone, methyl ethyl ketone, or combinations thereof. The volatile solvent system can include a mixture or combination of any of the volatile solvents set forth in the embodiments above. These volatile solvents should be chosen to be compatible with the rest of the formulation. It is desirable to use an appropriate weight percentage of the volatile solvent(s) in the formulation. Too much of the volatile solvent system prolongs the drying time. Too little of the volatile solvent system can make it difficult to spread the formulation on the skin. For most formulations, the weight percentage of the volatile solvent(s) can be from about 10 wt % to about 85 wt %, and more preferably from about 20 wt % to about 50 wt %.

The non-volatile solvent system can also be chosen or formulated to be compatible with the solidifying agent, the volatile solvent, and any other ingredients that may be present. For example, the solidifying agent can be chosen so that it is dispersible or soluble in the non-volatile solvent system. A desirable non-volatile solvent system can also act as a plasticizer for the solidifying agent. Most non-volatile solvent systems and solvent vehicles as a whole will be formulated appropriately only after experimentation. This being stated, non-volatile solvent(s) that can be used alone or in combination to form non-volatile solvent systems can be selected from a variety of pharmaceutically acceptable liquids. In one embodiment of the present invention, the non-volatile solvent system can include glycerol, propylene glycol, isostearic acid, oleic acid, propylene glycol, trolamine, tromethamine, triacetin, sorbitan monolaurate, sorbitan monooleate, sorbitan monopalmitate, butanol, or combinations thereof. In another embodiment, the non-volatile solvent system can include benzoic acid, butyl alcohol, dibutyl sebecate, diglycerides, dipropylene glycol, eugenol, fatty acids such as coconut oil, fish oil, palm oil, grape seed oil, isopropyl myristate, mineral oil, oleyl alcohol, vitamin E, triglycerides, sorbitan fatty acid surfactants, triethyl citrate, or combinations thereof. In a further embodiment, the non-volatile solvent system can include 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars ginger extract, glycerin, high fructose corn syrup, hydrogenated castor oil, IP palmitate, lemon oil, lime oil, limonene, milk, monoacetin, monoglycerides, nutmeg oil, octyidodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG Fatty acid esters such as PEG-stearate, PEG-oleate, PEG-laurate, PEG fatty acid diesters such as PEG-dioleate, PEG-distearate, PEG-castor oil, glyceryl behenate, PEG glycerol fatty acid esters such as PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers such as PEG-cetyl ether, PEG-stearyl ether, PEG-sorbitan fatty acid esters such as PEG-sorbitan diisosterate, PEG-sorbitan monostearate, propylene glycol fatty acid esters such as propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methylpyrrolidone, honey, polyoxyethylated glycerides, dimethyl sulfoxide, azone and related compounds, dimethylformamide, N-methyl formamide, fatty acid esters, fatty alcohol ethers, alkyl-amides (N,N-dimethylalkylamides), N-methylpyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, or combinations thereof. In yet a further embodiment, the non-volatile solvent system can include a combination or mixture of non-volatile solvents set forth in the any of the above discussed embodiments.

In addition to these and other considerations, the non-volatile solvent system can also serve as plasticizer in the adhesive formulation so that when the solidified layer is formed, the layer is flexible, stretchable, and/or otherwise “skin friendly.”

Certain volatile and/or nonvolatile solvent(s) that are irritating to the skin may be desirable to use to achieve the desired solubility and/or permeability of the drug. It is also desirable to add compounds that are both capable of preventing or reducing skin irritation and are compatible with the formulation. For example, in a formulation where the non-volatile and/or volatile solvent is capable of irritating the skin, it would be helpful to use a non-volatile solvent that is capable of reducing skin irritation. Examples of non-volatile solvents that are known to be capable of preventing or reducing skin irritation include, but are not limited to, glycerin, honey, and propylene glycol.

The formulations of the present invention may also contain ion-paring agents such as bases and acids. The purpose of these agent(s) can be to optimize the ionization state of the drug for obtaining desired delivery rates or to optimize the pH of the formulation or the skin tissues under the formulation layer to minimize irritation. Examples of suitable ion-pairing agents include, but are not limited to, trolamine, hydrochloric acid, sodium hydroxide, and/or acidic acid.

The selection of the solidifying agent can also be carried out in consideration of the other components present in the adhesive formulation. An appropriate solidifying agent is compatible with the formulation such that the formulation is in liquid or semi-liquid state (e.g. cream, paste, gel, ointment) without phase separation and without lumps or precipitation before any evaporation of the volatile solvent(s) and becomes a soft, coherent solid after the evaporation of at least some of the volatile solvent(s). The solidifying agent can be selected or formulated to be compatible to the drug and the solvent vehicle (including the volatile solvent(s) and the non-volatile solvent system), as well as provide desired physical properties to the solidified layer once it is formed. Depending on the drug, solvent vehicle, and/or other components that may be present, the solidifying agent can be selected from a variety of agents.

In one embodiment, the solidifying agent can include polyvinyl alcohol with a MW range of 20,000-70,000 (Amresco), esters of polyvinylmethylether/maleic anhydride copolymer (ISP Gantrez ES425 and Gantrez ES-225) with a MW range of 80,000-160,000, neutral copolymer of butyl methacrylate and methyl methacrylate (Degussa Plastoid B) with a MW range of 120,000-180,000, dimethylaminoethyl methacrylate-butyl methacrylate-methyl methacrylate copolymer (Degussa Eudragit E100) with a MW range of 100,000-200,000, ethyl acrylate-methyl methacrylate-trimethylammonioethyl methacrylate chloride copolymer with a MW greater than 5,000 or similar MW to Eudragit RLPO (Degussa), Zein (prolamine) with a MW greater than 5,000 such as Zein with a MW around 35,000 (Freeman industries), pregelatinized starch having a MW similar to Instant Pure-Cote B793 (Grain Processing Corporation), ethyl cellulose with a MW greater than 5,000 or a MW similar to Aqualon EC N7, N10, N14, N22, N50, or N100 (Hercules), fish gelatin having a MW 20,000-250,000 (Norland Products), gelatin, other animal sources with a MW greater than 5,000, acrylates/octylacrylamide copolymer with a MW greater than 5,000 or MW similar to National Starch, and/or Chemical Dermacryl 79.

In another embodiment, the solidifying agent can include ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, or combinations thereof. In a further embodiment, the solidifying agent can include ammonia methacrylate, carrageenan, cellulose acetate phthalate aqueous such as CAPNF from Eastman, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers such as BASF's Kollicoat polymers, including polyhydroxyethyl methacrylate, poly(methacrylic acid) copolymers and methylmethacrylate copolymers or combinations thereof. In another embodiment, the solidifying agent can include a combination of solidifying agents set forth in the any of the above discussed embodiments. Other polymers may also be suitable as the solidifying agent, depending on the solvent vehicle components, the drug, and the specific functional requirements of the given formulation. In one embodiment, the solidifying agent can include a polyurethane.

In some embodiments of the present invention, it may be desirable to add an additional agent or substance to the formulation so as to provide enhanced or increased adhesive characteristics. The additional adhesive agent or substance can be an additional non-volatile solvent or an additional solidifying agent. Non-limiting examples of substances which might be used as additional adhesion enhancing agents include copolymers of methylvinyl ether and maleic anhydride (Gantrez polymers), polyethylene glycol and polyvinyl pyrrolidone, gelatin, low molecular weight polyisobutylene rubber, copolymer of acrylsan alkyl/octylacrylamido (Dermacryl 79), various aliphatic resins and aromatic resins, and combinations thereof.

The non-volatile solvent system and the solidifying agent should be compatible with one another. Compatibility can be defined as i) the solidifying agent does not substantially negatively influence the function of the non-volatile solvent system, though some reduction of flux may be acceptable; ii) the solidifying agent can hold the non-volatile solvent system in the solidified layer so that substantially no non-volatile solvent oozes out of the layer, and/or iii) the solidified layer formed with the selected non-volatile solvent system and the solidifying agent has acceptable flexibility, rigidity, tensile strength, elasticity, and adhesiveness. The weight ratio of the non-volatile solvent system to the solidifying agent can be from about 0.1:1 to about 10:1. In another aspect, the ratio between the non-volatile solvent system and the solidifying agent can be from about 0.5:1 to about 2:1.

The thickness of the formulation layer applied on the skin should also be appropriate for a given formulation and desired drug delivery considerations. If the layer is too thin, the amount of the drug may not be sufficient to support sustained delivery over the desired length of time. If the layer is too thick, it may take too long to form a non-messy outer surface while the solidified layer is forming. If the drug is very potent and the peel has very high tensile strength, a layer as thin as about 0.01 mm may be sufficient. If the drug has rather low potency and the solidified layer has low tensile strength, a layer as thick as about 2-3 mm maybe needed. Thus, for most drugs and formulations, the appropriate thickness can be from about 0.01 mm to about 3 mm, but more typically, from about 0.05 mm to about 1 mm.

The flexibility and stretchability of a solidified layer, which is optionally peelable, can be desirable in some applications. For instance, the formulation may be used to treat a skin area that is suffering from neuropathy and is directly over joints and muscles. Skin areas over joints and certain muscle groups are often significantly stretched during body movements. Such movement prevents non-stretchable patches from maintaining good skin contact. Lotions, ointments, creams, gels, pastes, or the like also may not be suitable for use for the reasons cited above. As such, a flexible, elastic solidified layer will be desirable in these applications can offer unique advantages and benefits. It should be pointed out that although good stretchability can be desirable in many applications, the solidifying formulations of the present invention do not always need to be stretchable, as certain applications of the present invention do not necessarily benefit from this property and these solidified layers are also included in accordance with embodiments of the present invention.

A further feature of the formulations of the present invention is related to the drying time. If a formulation dries too quickly, the user may not have sufficient time to spread the formulation into a thin layer on the skin surface before the formulation is solidified, leading to poor skin contact. If the formulation dries too slowly, the user may have to wait a long time before resuming normal activities (e.g. putting clothing on) that may remove un-solidified formulation. Thus, it is desirable for the drying time to be longer than about 15 seconds but shorter than about 15 minutes, and preferably from about 0.5 minutes to about 5 minutes.

Another feature of the formulations of the present invention is related to solidifying formulations comprising a drug for treating neuropathy, a non-volatile solvent system comprising at least one non-volatile solvent, a solidifying agent, and a volatile solvent system comprising a volatile solvent whose boiling point is below 20° C. (such a solvent can be used as a propellant or can be dissolved in the formulation). In one embodiment, the formulation can be stored in a pressurized container and be sprayed on the skin surface with the help of the propellant. Some hydrofluorocarbons commonly used as propellants in pharmaceutical or dosmetic industries can work in this design. More specifically, the propellants may include, but not limited to dimethyl ether, butane, 1,1, Difluoroethane, 1,1,1,2 tetrafluorethane, 1,1,1,2,3,3,3-heptafluoropropane, 1,1,1,3,3,3 hexafluoropropane, or a mixture thereof. The formulation may also be expelled out of the container and applied on the skin via a manual pump. Formulations comprising room temperature gaseous volatile solvents are expected to dry much faster. Spraying the formulation onto the skin suffering from neuropathy can avoid touching the skin with an applicator which can cause severe pain in the sometimes hypersensitive skin.

The formulations of the present invention may further comprise a pH modifying agent for adjusting the pH of the formulation to a point or a range most suitable for the delivery of minoxidil while being skin friendly.

Other benefits of the solidified layers of the present invention include the presence of a physical barrier that can be formed by the material itself. Since many subjects suffering from neuropathy may feel pain when their skin area is touched with minimal pressure, the physical barrier of the solidified layer can prevent or minimize pain caused by accidental contact.

The adhesion to skin and elasticity of the material is such that the solidified layer may not easily separate from the skin. For example, in one embodiment, the solidified layer can be stretched in at least one direction by up to about 5% or even 10% or more without cracking, breaking, or separating form a skin surface to which the solidified layer is applied.

These and other advantages can be summarized in accordance with some embodiments as follows. The solidifying formulation of the present invention can be in an initial form that is easy to apply as a semisolid dosage form. Additionally, upon volatile solvent system evaporation, the dosage form is relatively thick and can contain much more active drug than a typical layer of traditional cream, gel, lotion, ointment, paste, etc., and further, is not as subject to unintentional removal. After the evaporation of the volatile solvent(s) and the formation of the solidified layer, the drug in the remaining formulation can be delivered at desired delivery rates over sustained periods of time. Further, as the solidified layer remains adhered to skin and is preferably peelable, easy removal of the solidified layer can occur, usually without the aid of a solvent or surfactant. In some embodiments, the non-volatile solvent system is so selected or formulated that it is flux-enabling for the drug. In some embodiments, the adhesion to skin and elasticity of the material is such that the solidified layer will not separate from the skin upon skin stretching at highly stretchable skin areas, such as over joints and muscles. For example, in one embodiment, the solidified layer can be stretched to 10% or greater in one direction without cracking, breaking, and/or separating form a skin surface to which the solidified layer is applied. Still further, the solidified layer can be configured to advantageously deliver drug and protect sensitive skin areas without cracking or breaking.

As a further note, it is a unique feature that the solidified layers of the present invention can keep a substantial amount of the non-volatile solvent system, which is optimized for delivering the drug, on the body surface. This feature can provide unique advantages over existing products. For example, in some semi-solid formulations, upon application to a skin surface the volatile solvents quickly evaporate and the formulation layer solidifies into a hard lacquer-like layer. The drug molecules are immobilized in the hard lacquer layer and are substantially unavailable for delivery into the skin surface. As a result, it is believed that the delivery of the drug is not sustained over a long period of time. In contrast to this type of formulation, the solidified layers formed using the formulations of the present invention keep the drug molecules quite mobile in the non-volatile solvent system which is in contact with the skin surface, thus ensuring sustained delivery.

Solidifying formulations of the present invention that comprise minoxidil and at least one other drug may provide additional benefits. For example, a formulation for treating neuropathy in accordance with the present invention may include minoxidil and a local anesthetic such as lidocaine and/or tetracaine. The local anesthetic can be present in either the salt form or in the base form. Preferably, the non-volatile solvent system includes at least one of propylene glycol and dipropylene glycol, and isostearic acid. Similar formulations may comprise other minoxidil in combination with other drugs, such as amitriptyline and ketamine, amitriptyline and a local anesthetic, etc.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

Human epidermal membrane (HEM) is used as the model membrane for the in vitro flux studies described in herein. Previously prepared human epidermal membrane samples are mounted carefully between the donor and receiver chambers of a Franz diffusion cell. The receiver chamber is filled with pH 7.4 phosphate buffered saline (PBS). The experiment is initiated by placing test formulations (of Examples 2-3 and 5-6) on the stratum corneum (SC) of the skin sample. Franz cells are placed in a heating block maintained at 37° C. and the HEM temperature is maintained at 35° C. At predetermined time intervals, the entire volume of the receiver chamber is withdrawn and replaced with fresh PBS solution. An aliquot of the removed receiver solution is analyzed for drug content for skin flux determination. Skin flux (μg/cm²/h) is determined from the steady-state slope of a plot of the cumulative amount of permeation versus time.

### Examples 2-3

Solidifying formulations for treating diabetic neuropathy and the associated neuropathic pain are prepared and a qualitative assessment of peel flexibility and viscosity is performed. The formulation components are presented in Table 1 below.

TABLE 1

Example

| | |
|---|---|
| 2 | 3 |

| | |
|---|---|
| Components | Parts by Weight |

| | | |
|---|---|---|
| Polyvinyl alcohol | 22.2 | 22.9 |
| (Mw 30,000-50,000) | | |
| Water | 44.4 | 45.7 |
| Propylene glycol | 22.2 | 20.2 |
| Ethanol | 4.4 | 4.0 |
| 5M HCl solution | 1.8 | N/A |
| Isostearic Acid (ISA) | N/A | 2.2 |
| Minoxidil | 5 | 5 |

The peel formulation in Examples 2 and 3 have a viscosity that may be desirable for application on a skin surface. A solidified peel is formed when Examples 2 and 3 are spread on a silicone coated polyester release liner and the solidified peels are stretchable by 5% in one direction without cracking or splitting. Peel formulations of Examples 2-3 are prepared in the following manner:
The peel forming agents (solidifying agents) are dissolved in the volatile solvent (i.e. dissolve polyvinyl alcohol in water).
An adequate non-volatile solvent (propylene glycol for Example 2 and in the case of Example 3 propylene glycol and isostearic acid) and additional volatile solvent, ethanol, is mixed with the peel forming agent/volatile solvent mixture.
The resulting solution is vigorously mixed well for several minutes.
Drug is then added and the solidifying formulation is mixed again for several minutes followed by addition of HCl solution for pH adjustment Example 2.

### Example 4

The solidifying formulations of Examples 2-3 are tested in a HEM in vitro model as described in Example 1. Table 2 shows data obtained using the experimental process outlined above.

TABLE 2
Steady-state flux (J)

| | J* |
|---|---|
| Formulation | (μg/cm²/h) |
| Rogaine 5% Minoxidil Solution | 0.8 ± 0.1 |
| Example 2 | 0.85 ± 0.03 |
| Example 3 | 4.2 ± 0.5 |

*Skin flux measurements represent the mean and st. dev of three determinations.

Regarding the formulation described in Examples 2 and 3, ethanol and water make up the volatile solvent system, and the propylene glycol (Example 2) and the ISA/propylene glycol mixture (Example 3) make up the non-volatile solvent system. Through experimentation, it was determined that ISA and propylene glycol can be used together to provide the desirable solubility for the minoxidil, while being compatible with the polyvinyl alcohol (solidifying agent, or peel-forming agent). Further, in this embodiment, propylene glycol also serves as a plasticizer in the formulation after the ethanol and water (volatile solvent) has evaporated. The steady state flux of minoxidil from formulation Examples 2 and 3 demonstrate the significance of the non-volatile solvent in dictating the flux-generating power of the entire formulation.

### Examples 5-6

Solidifying formulations for treating diabetic neuropathy are prepared and a qualitative assessment of peel flexibility and viscosity are evaluated. The formulation components are presented in Table 3 below.

TABLE 3

Example

| | |
|---|---|
| 5 | 6 |

| | |
|---|---|
| Components | Parts by Weight |

| | | |
|---|---|---|
| Polyurethane resin | 14 | 15 |
| Water | 8 | 7 |
| Propylene glycol | 14 | 12 |
| Ethanol | 57 | 59 |
| 5M HCl solution | 2 | N/A |
| Isostearic Acid | N/A | 2 |
| Minoxidil | 5 | 5 |

The solidifying formulation in Examples 5 and 6 have a viscosity that may be desirable for application on a skin surface. A solidified peel is form when Examples 5 and 6 are spread on a silicone coated polyester release liner and the solidified peels are stretchable by 5% in one direction without cracking or splitting. Peel formulations of Examples 5-6 are prepared in the following manner:
The peel forming agents are dissolved in the volatile solvent (i.e., dissolve polyurethane in ethanol).
An adequate non-volatile solvent (propylene glycol for Example 5 and in the case of Example 6 propylene glycol and isostearic acid) and additional volatile solvent, water, is mixed with the peel forming agent/volatile solvent mixture.
The resulting solution is vigorously mixed well for several minutes.
Drug is then added and the solidifying formulation is mixed again for several minutes followed by addition of HCl solution for pH adjustment in Example 5.

### Example 7

The solidifying formulations of Examples 5-6 are tested in a HEM in vitro model described in Example 1. Table 4 shows data obtained using the experimental process outlined above.

TABLE 4
Steady-state flux (J)

| | J |
|---|---|
| Formulation | (μg/cm²/h) |
| Rogaine 5% Minoxidil Solution* | 0.8 ± 0.1 |
| Example 5* | 1.2 ± 0.1 |
| Example 6** | 18.1 ± 0.2 |

*Skin flux measurements represent the mean and st. dev of three determinations.
**Flux measurement represents the mean and st dev of two determinations.

Regarding the formulation described in Examples 5 and 6, ethanol is the volatile solvent system, and the propylene glycol (Example 2) and the ISA/propylene glycol mixture (Example 3) make up the non-volatile solvent system. Through experimentation, it was determined that ISA and propylene glycol used together provide desirable solubility for the drug, while being compatible with the polyurethane film former. Further, in this embodiment, propylene glycol serves as a plasticizer in the peelable formulation after the ethanol (volatile solvent) has evaporated. The steady state flux of minoxidil from formulation Examples 5 and 6 demonstrate the significance of the non-volatile solvent in dictating the flux-generating power of the entire formulation.

### Example 8

A solidifying formulation similar to the Examples of 2-3 is prepared, except that the peel formulation contains an emulsifying agent such as acacia, or crosslinked polymers of acrylic acid (e.g., Carbopol polymers, Pemulen), or mixtures of surfactants with the appropriate HLB number to effectively suspend a hydrophobic non-volatile solvent in a non-miscible volatile system.

### Example 9

A solidifying formulation similar to that taught in Examples of 2-3, and 5-6 is prepared, except that the peel formulation contains an additional tackifying polymer selected from one or more of copolymers of methylvinyl ether and maleic anhydride (Gantrez) and polyvinylpyrrolidone, gelatin, low molecular weight polyisobtylene rubber, copolymers of acrylsan alkyl/octylacrylamido, aliphatic resins, and/or aromatic resins. The tackifying polymers are added into the formulation to improve the wear properties of the formulation on the skin surface.

### Example 10

A solidifying formulation similar to that taught in Examples of 2-3, and 5-6 is prepared, except that the peel formulation contains at least one film forming polymer selected from polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine (Zein), pregelatinized starch, ethyl cellulose, fish gelatin, gelatin, acrylates/octylacrylamide copolymers, ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and/or polyhydroxyethyl methacrylate.

### Example 11

A solidifying formulation similar to that taught in Examples of 2-3, and 5-6 is prepared, except that the peel formulation contains at least one non-volatile solvent selected from tetrahydroxypropyl ethylenediamine, triacetin, span 20, isostearic acid, glycerin, propylene glycol, dipropylene glycol glycerol, propylene glycol, isostearic acid, oleic acid, propylene glycol, trolamine, tromethamine, triacetin, sorbitan monolaurate, sorbitan monooleate, sorbitan monopalmitate, butanol, 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars, ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methylpyrrolidone, honey, polyoxyethylated glycerides, dimethyl sulfoxide, azone and related compounds, dimethylformamide, N-methyl formamide, fatty acid esters, fatty alcohol ethers, alkyl-amides (N,N-dimethylalkylamides), N-methylpyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50, shea butter, aloe vera juice, phenyl trimethicone, and/or hydrolyzed wheat protein.

### Example 12

A solidifying formulation similar to that taught in Examples of 2-3, and 5-6 is prepared, except that the peel formulation contains at least one volatile solvent selected from ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, iso-amyl acetate, denatured alcohol, methanol, propanol, isobutene, pentane, hexane, chlorobutanol, turpentine, cytopentasiloxane, cyclomethicone, methyl ethyl ketone, and/or mixtures with water thereof.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A formulation for treating neuropathy, comprising:
a) an amount of minoxidil suitable for treating neuropathy;
b) a solvent vehicle, comprising:
i) a volatile solvent system including at least one volatile solvent, and
ii) a non-volatile solvent system including at least one non-volatile solvent, wherein the non-volatile solvent system facilitates transdermal delivery of the minoxidil at a therapeutically effective rate over a sustained period of time; and
c) a solidifying agent,
wherein the formulation has a viscosity suitable for application and adhesion to a skin surface prior to evaporation of the volatile solvent system, wherein the formulation applied to the skin surface forms a solidified layer after at least partial evaporation of the volatile solvent system, and wherein the minoxidil continues to be dermally delivered at the therapeutically effective rate after the volatile solvent system is at least substantially evaporated.

2. A formulation as in claim 1, wherein the neuropathy is caused by diabetes.

3. A formulation as in claim 2, wherein the diabetic neuropathy causes pain.

4. A formulation as in claim 1, wherein the non-volatile solvent system is flux-enabling for the minoxidil.

5. A formulation as in claim 1, wherein the non-volatile solvent system acts as a plasticizer for the solidifying agent.

6. A formulation as in claim 1, wherein the formulation further comprises a pH modifying agent.

7. A formulation as in claim 1, wherein the volatile solvent system comprises water.

8. A formulation as in claim 1, wherein the volatile solvent system is substantially free of water.

9. A formulation as in claim 1, wherein the volatile solvent system comprises at least one member selected from the group of ethanol, isopropyl alcohol, and combinations thereof.

10. A formulation as in claim 1, wherein the volatile solvent system comprises at least one solvent more volatile than water, and includes a member selected from the group consisting of ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, and combinations thereof.

11. A formulation as in claim 1, wherein the volatile solvent system comprises at least one solvent more volatile than water, and includes a member selected from the group consisting of iso-amyl acetate, denatured alcohol, methanol, propanol, isobutene, pentane, hexane, chlorobutanol, turpentine, cytopentasiloxane, cyclomethicone, methyl ethyl ketone, and combinations thereof.

12. A formulation as in claim 1, wherein the non-volatile solvent system comprises at least one solvent selected from the group consisting of tetrahydroxypropyl ethylenediamine, triacetin, span 20, isostearic acid, glycerin, propylene glycol, dipropylene glycol, and combinations thereof.

13. A formulation as in claim 1, wherein the non-volatile solvent system comprises at least one solvent selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

14. A formulation as in claim 1, wherein the non-volatile solvent system comprises at least one solvent selected from the group consisting of benzoic acid, butyl alcohol, dibutyl sebecate, diglycerides, dipropylene glycol, eugenol, fatty acids, isopropyl myristate, mineral oil, oleyl alcohol, vitamin E, triglycerides, sorbitan fatty acid surfactants, triethyl citrate, and combinations thereof.

15. A formulation as in claim 1, wherein the non-volatile solvent system comprises at least one solvent selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars, ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methylpyrrolidone, honey, polyoxyethylated glycerides, dimethyl sulfoxide, azone and related compounds, dimethylformamide, N-methyl formamide, fatty acid esters, fatty alcohol ethers, alkyl-amides (N,N-dimethylalkylamides), N-methylpyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50, shea butter, aloe vera juice, phenyl trimethicone, hydrolyzed wheat protein, and combinations thereof.

16. A formulation as in claim 1, wherein the non-volitile solvent system includes isostearic acid.

17. A formulation as in claim 1, wherein the non-volitile solvent system includes oleic acid.

18. A formulation as in claim 1, wherein non-volitile solvent system includes propylene glycol.

19. A formulation as in claim 1, wherein the solidifying agent includes at least one member selected from the group consisting of polyvinyl alcohol, polyurethane, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine (Zein), pregelatinized starch, ethyl cellulose, fish gelatin, gelatin, acrylates/octylacrylamide copolymers, polyhydroxyethyl methacrylate, polyurethane, and combinations thereof.

20. A formulation as in claim 1, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, polyurethane, and combinations thereof.

21. A formulation as in claim 1, wherein the solidifying agent includes at least one member selected from the group consisting of ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

22. A formulation as in claim 1, wherein the formulation includes polyurethane.

23. A formulation as in claim 1, wherein the formulation includes a second drug.

24. A formulation as in claim 23, wherein the second drug includes at least one member selected from the group consisting of local anesthetics, steroids, alpha-2 agonists, tricyclic anti-depressants, antiviral drugs, 5-HT2A receptor antagonists, and combinations thereof.

25. A formulation as in claim 23, wherein the second drug is a local anesthetic selected from the group consisting of lidocaine, bupivacaine, ropivacaine, tetracaine, and combinations thereof.

26. A formulation as in claim 25, wherein the local anesthetic is in free base form.

27. A formulation as in claim 23, wherein the second drug includes dexamethasone.

28. A formulation as in claim 23, wherein the second drug includes clonidine.

29. A formulation as in claim 23, wherein the second drug is a local anesthetic agent and the non-volatile solvent system is capable of generating a flux of the local anesthetic of at least 5 mcg/cm²/h.

30. A formulation as in claim 1, wherein the solidified layer is sufficiently flexible and adhesive to the skin such that when applied to the skin at a human joint, the solidified layer will remain substantially intact on the skin upon bending of the joint.

31. A formulation as in claim 1, wherein the volatile solvent system comprises a volatile solvent whose boiling point is below 20° C.

32. A formulation as in claim 31, wherein the volatile solvent with the boiling point below 20° C. is completely dissolved in the formulation.

33. A formulation as in claim 31, wherein the volatile solvent with the boiling point below 20° C. is included in the formulation as a propellant for pressurized spray-on application.

34. A formulation as in claim 31, wherein the volatile solvent with the boiling point below 20° C. is a hydrofluorocarbon.

35. The formulation as in claim 31, wherein the volatile solvent whose boiling point is below 20° C. is selected from the group consisting of dimethyl ether, butane, 1,1, difluoroethane, 1,1,1,2 tetrafluorethane, 1,1,1,2,3,3,3-heptafluoropropane, 1,1,1,3,3,3 hexafluoropropane, and combinations thereof.

36. A formulation as in claim 1, wherein the formulation is formulated to deliver the minoxidil at therapeutically effective rates for at least about 2 hours following the formation of the solidified layer.

37. A formulation as in claim 1, wherein the formulation is formulated to deliver the minoxidil at therapeutically effective rates for at least about 4 hours following the formation of the solidified layer.

38. A formulation as in claim 1, wherein the formulation is formulated to deliver the minoxidil at therapeutically effective rates for at least about 8 hours following the formation of the solidified layer.

39. A formulation as in claim 1, wherein the formulation is formulated to deliver the minoxidil at therapeutically effective rates for at least about 12 hours following the formation of the solidified layer.

40. A formulation as in claim 1, wherein the solidifying agent is dispersed in the solvent vehicle.

41. A formulation as in claim 1, wherein solidifying agent is solvated in the solvent vehicle.

42. A formulation as in claim 1, wherein the weight ratio of the non-volatile solvent system to the solidifying agent is from about 0.1:1 to about 10:1.

43. A formulation as in claim 1, wherein the weight ratio of the non-volatile solvent system to the solidifying agent is from about 0.5:1 to about 2:1.

44. A formulation as in claim 1, wherein the non-volatile solvent system is capable of causing human skin irritation and at least one non-volatile solvent of the non-volatile solvent system is capable of reducing the skin irritation.

45. A formulation as in claim 44, wherein the non-volatile solvent capable of reducing skin irritation includes member selected from the group consisting of glycerin, propylene glycol, honey, and combinations thereof.

46. A formulation as in claim 1, wherein the solidified layer is formed within about 15 minutes of application to the skin surface under standard skin and ambient conditions.

47. A formulation as in claim 1, wherein the solidified layer is formed within about 5 minutes of the application to the skin surface under standard skin and ambient conditions.

48. A formulation as in claim 1, wherein the formulation has an initial viscosity prior to skin application from about 100 to about 3,000,000 centipoises.

49. A formulation as in claim 1, wherein the formulation has an initial viscosity prior to skin application from about 1,000 to about 1,000,000 centipoises.

50. A formulation as in claim 1, wherein the weight percentage of the volatile solvent system is from about 10 wt % to about 85 wt %.

51. A formulation as in claim 1, wherein the weight percentage of the volatile solvent system is from about 20 wt % to about 50 wt %.

52. A formulation as in claim 1, wherein the non-volatile solvent system includes multiple non-volatile solvents, and at least one of the non-volatile solvents is capable of improving the compatibility of the non-volatile solvent system with the solidifying agent.

53. A formulation as in claim 1, wherein the formulation further comprises an additional agent that increases adhesion of the formulation when applied to a body surface.

54. A formulation as in claim 53, wherein the additional agent includes a member selected from the group consisting of copolymers of methylvinyl ether and maleic anhydride, polyethylene glycol and polyvinyl pyrrolidone, gelatin, low molecular weight polyisobutylene rubber, copolymer of acrylsan alkyl/octylacrylamido, aliphatic resins, aromatic resins, and combinations thereof.

55. A formulation as in claim 1, wherein the solidified layer, upon formation, is a soft, coherent sold that is peelable from a skin surface as a single piece or as only a few large pieces relative to the application size.

56. A formulation as in claim 1, wherein the minoxidil is comprises about 0.1 wt % to about 30 wt % of the total of formulation.

57. A formulation as in claim 1, wherein the minoxidil comprises about 0.5 wt % to about 10 wt % of the formulation.

58. A formulation as in claim 1, wherein the minoxidil comprises about 2 wt % to about 6 wt % of the formulation.

59. A formulation as in claim 1, wherein the solidified layer transdermally delivers the minoxidil at a transdermal flux rate of at least 0.02 μg/cm²/hour across human skin.

60. A formulation as in claim 1, wherein the solidified layer transdermally delivers the minoxidil at a transdmeral flux rate of about 0.1 μg/cm²/hour to about 5.0 μg/cm²/hour across human skin.

61. A formulation as in claim 1, wherein the solidified layer transdermally delivers the minoxidil at a transdmeral flux rate of about 0.4 μg/cm²/hour to about 4.0 μg/cm²/hour across human skin.

62. A method for treating neuropathy, comprising:
a) applying a solidifying formulation to a skin surface of a subject suffering from neuropathy, the solidifying formulation comprising:
i) an amount of minoxidil suitable for treating neuropathy,
ii) a solvent vehicle, comprising:
a volatile solvent system including at least one volatile solvent, and
a non-volatile solvent system including at least one non-volatile solvent, wherein the non-volatile solvent system is capable of facilitating transdermal delivery of the minoxidil, and
iii) a solidifying agent,
wherein the formulation has a viscosity suitable for application and adhesion to a skin surface prior to evaporation of the volatile solvent system;
b) solidifying the formulation to form a solidified layer on the skin surface by at least partial evaporation of the volatile solvent system; and
c) dermally delivering the minoxidil from the solidified layer to the subject at therapeutically effective rates over a sustained period of time to treat the neuropathy.

63. A method as in claim 62, wherein the neuropathy is caused by diabetes.

64. A method as in claim 62, wherein the step of applying includes applying the formulation at a thickness from about 0.01 mm to about 3 mm.

65. A method as in claim 62, wherein the step of applying includes applying the formulation at a thickness from about 0.05 mm to about 1 mm.

66. A method as in claim 62, wherein the volatile solvent system comprises water.

67. A method as in claim 62, wherein the volatile solvent system comprises ethanol, propanol, or a combination thereof.

68. A method as in claim 62, wherein the volatile solvent system includes at least one member selected from the group consisting of ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone and combinations thereof.

69. A method as in claim 62, wherein the volatile solvent system includes at least one member selected from the group consisting of iso-amyl acetate, denatured alcohol, methanol, propanol, isobutene, pentane, hexane, chlorobutanol, turpentine, cytopentasiloxane, cyclomethicone, methyl ethyl ketone, and combinations thereof.

70. A method as in claim 62, wherein the non-volatile solvent system comprises at least one solvent including a member selected from the group consisting of tetrahydroxypropyl ethylenediamine, triacetin, span 20, isostearic acid, glycerin, propylene glycol, dipropylene glycol, or a mixture thereof.

71. A method as in claim 62, wherein the non-volatile solvent system includes at least one member selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

72. A method as in claim 62, wherein the non-volatile solvent system includes at least one member selected from the group consisting of benzoic acid, butyl alcohol, dibutyl sebecate, diglycerides, dipropylene glycol, eugenol, fatty acids, isopropyl myristate, mineral oil, oleyl alcohol, vitamin E, triglycerides, sorbitan fatty acid surfactants, triethyl citrate, and combinations thereof.

73. A method as in claim 62, wherein the non-volatile solvent system includes at least one member selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars, ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methylpyrrolidone, honey, polyoxyethylated glycerides, dimethyl sulfoxide, azone and related compounds, dimethylformamide, N-methyl formamide, fatty acid esters, fatty alcohol ethers, alkyl-amides (N,N-dimethylalkylamides), N-methylpyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof.

74. A method as in claim 62, wherein the solidifying agent includes at least one member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine (Zein), pregelatinized starch, ethyl cellulose, fish gelatin, gelatin, acrylates/octylacrylamide copolymers, polyhydroxyethyl methacrylate, polyurethane, and combinations thereof.

75. A method as in claim 62, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate and combinations thereof.

76. A method as in claim 62, wherein the solidifying agent includes at least one member selected from the group consisting of ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

77. A method as in claim 62, wherein the adhesive formulation further includes a second drug.

78. A method as in claim 77, wherein the second drug includes at least one member selected from the group consisting of local anesthetics including tetracaine, lidocaine, ropivacaine, steroids, alpha-2 agonists, tricyclic anti-depressants including amitriptyline, anticonvulsants, antiviral drugs including acyclovir, penciolovir, famciclovir, and valacyclovir, 5-HT2A receptor antagonists including ketanserin, N-methyl-D-aspartate antagonists, ketamine, and combinations thereof.

79. A method as in claim 62, wherein the solidified layer is sufficiently flexible and adhesive to the skin such that when applied to the skin at a human joint, the solidified layer will remain substantially intact on the skin upon bending of the joint.

80. A method as in claim 62, wherein the formulation is left on the skin for at least about 2 hours following the formation of the solidified layer.

81. A method as in claim 62, wherein the formulation is left on the skin for at least about 8 hours following the formation of the solidified layer.

82. A method as in claim 62, wherein the weight ratio of the non-volatile solvent system to the solidifying agent is from about 0.5:1 to about 2:1.

83. A method as in claim 62, wherein the solidified layer is formed within 15 minutes of application to the skin surface under standard skin and ambient conditions.

84. A method as in claim 62, wherein the formulation is sprayed on the skin.

85. A method as in claim 62, wherein the formulation is applied on the skin using a manual pump.

86. A method as in claim 62, wherein the formulation has an initial viscosity prior to skin application from about 100 to about 3,000,000 centipoises.

87. A method as in claim 62, wherein the weight percentage of the volatile solvent system is from about 10 wt % to about 85 wt %.

88. A method as in claim 62, further comprising the step of peeling the solidified layer from the skin after the sustained period of time to remove the solidified layer.

89. A method as in claim 62, further comprising the step of washing the solidified layer form the skin using a solvent after the sustained period of time to remove the solidified layer.

90. A method as in claim 62, wherein the neuropathy is associated with shingles.

91. A method as in claim 62, wherein the neuropathy is associated with diabetes.

92. A method as in claim 62, wherein the neuropathy is associated with postherpatic neuralgia.

93. A method as in claim 62, wherein the neuropathy is associated with postsurgical or post-traumatic pain.

94. A method as in claim 62, wherein the minoxidil is comprises 0.1 wt % to 30 wt % of the total of formulation.

95. A method as in claim 62, wherein the minoxidil comprises about 0.5 wt % to about 10 wt % of the formulation.

96. A method as in claim 62, wherein the minoxidil comprises about 2 wt % to about 6 wt % of the formulation.

97. A method as in claim 62, wherein the solidified layer transdermally delivers the minoxidil at a transdermal flux rate of at least 0.02 μg/cm²/hour across human skin.

98. A method as in claim 62, wherein the solidified layer transdermally delivers the minoxidil at a transdmeral flux rate of about 0.1 μg/cm²/hour to about 5.0 μg/cm²/hour across human skin.

99. A method as in claim 62, wherein the solidified layer transdermally delivers the minoxidil at a transdmeral flux rate of about 0.4 μg/cm²/hour to about 4.0 μg/cm²/hour across human skin.

100. A solidified layer for delivering a drug for treating neuropathy, comprising:
a) an amount of minoxidil suitable for treating neuropathy;
b) a non-volatile solvent system suitable for providing transdermal flux of the minoxidil; and
c) a solidifying agent,
wherein the solidified layer is stretchable by 5% in one direction without cracking, breaking, and/or separating from a skin surface to which the layer is applied.

101. A solidified layer as in claim 100, wherein the solidified layer is sufficiently adhesive and flexible to remain substantially intact on standard skin under standard testing condition for at least about 2 hours.

102. A solidified layer as in claim 100, wherein the weight ratio of the non-volatile solvent system to the solidifying agent is from about 0.1:1 to about 10:1.

103. A solidified layer as in claim 100, wherein the solidified layer is formed within 15 minutes of the application to the skin surface under standard skin and ambient conditions.

104. A solidified layer as in claim 100, wherein the solidified layer further includes a second drug selected from the group consisting of local anesthetics, steroids, alpha-2 agonists, tricyclic anti-depressants, antiviral drugs, 5-HT2A receptor antagonists, and combinations thereof.

105. A solidified layer as in claim 100, wherein the non-volatile solvent system acts as a plasticizer for the solidifying agent.

106. A solidified layer as in claim 100, wherein the solidified layer can be removed by washing.

107. A solidified layer as in claim 100, wherein the solidified layer is a peel and can be removed by peeling from the skin surface as a single piece or as only a few large pieces relative to the application size.

108. A solidified layer as in claim 100, wherein the solidified layer is flux-enabling for the minoxidil.

109. A solidified layer as in claim 100, wherein the solidified layer is adhesive to the skin surface on one surface, and is non-adhesive on an opposing surface.

110. A solidified layer as in claim 100, wherein the solidified layer is formulated to deliver a majority the minoxidil that is dermally deliverable therefrom while the solidified layer is substantially devoid of water and any solvent more volatile than water.

111. A solidified layer as in claim 100, wherein the solidified layer transdermally delivers the minoxidil at a transdermal flux rate of at least 0.02 μg/cm²/hour across human skin.

112. A solidified layer as in claim 100, wherein the solidified layer transdermally delivers the minoxidil at a transdmeral flux rate of about 0.1 μg/cm²/hour to about 5.0 μg/cm²/hour across human skin.

113. A solidified layer as in claim 100, wherein the solidified layer transdermally delivers the minoxidil at a transdmeral flux rate of about 0.4 μg/cm²/hour to about 4.0 μg/cm²/hour across human skin.

114. A solidified layer as in claim 100, wherein the neuropathy is caused by diabetes.
